(19) United States
(12) Patent Application Publication
(10) Pub. No.: US 2026/0071011 A1
(43) Pub. Date: Mar. 12, 2026

(54) COMPOUND, RESIN COMPOSITION, PREPREG, RESIN FILM, METAL-CLAD LAMINATE, PRINTED WIRING BOARD, AND SEMICONDUCTOR PACKAGE

(71) Applicant: Resonac Corporation, Tokyo (JP)

(72) Inventors: Ryuji AKEBI, Tokyo (JP); Keiichi KASUGA, Tokyo (JP)

(21) Appl. No.: 19/106,474

(22) PCT Filed: Apr. 22, 2024
(86) PCT No.: PCT/JP2024/015718

§ 371 Date: Feb. 25, 2025

(30) Foreign Application Priority Data
Jun. 16, 2023 (JP) 2023-099182

(51) Int. Cl. C08F 12/12; C07C 13/465; C08J 5/18; C08J 5/24; C09J 145/00; H01L 23/14; H01L 23/29

(52) U.S. Cl. CPC C08F 12/12; C07C 13/465; C08J 5/18; C08J 5/24; C09J 145/00; H10W 70/695; H10W 74/47; C07C 2602/08; C08J 2345/00

(57) ABSTRACT

An embodiment relates to a compound including: an indene ring; a vinylbenzyl group; and an aromatic ring-containing group of 6 or more carbon atoms without a polymerizable carbon-carbon double bond, in which both of the vinylbenzyl group and the aromatic ring-containing group are directly bonded to the indene ring.

### TECHNICAL FIELD

The present disclosure relates to a compound, a resin composition, a prepreg, a resin film, a metal-clad laminate, a printed wiring board, and a semiconductor package.

### BACKGROUND ART

Metal-clad laminates represented by copper-clad laminates, prepregs usable for metal-clad laminates, and semiconductor packages using metal-clad laminates are used in mobile communication devices such as smartphones, base station devices thereof, network infrastructure devices such as servers, routers, and large servers, and various electronic devices such as large computers, personal computers, and industrial computers. They are also used in electronic devices mounted in household appliances and automobiles. From thereamong, due to the spread of 5G, demand for electronic communication devices to process a large amount of data at a high speed is increasing.

When an electronic device processes a large amount of data at a high speed, a substrate material with a small transmission loss in a high frequency range is required for the device. A resin with a low dielectric constant and a low dielectric loss tangent is used for a substrate material to provide a substrates with a low transmission loss. However, with the development of communication technology in recent years, further development of a resin with a low dielectric constant and a low dielectric loss tangent is required.

Patent Literature 1 discloses a compound with an indene ring structure, into which a vinylbenzyl group is introduced as a curable vinylbenzyl compound which can be an excellent cured product with a low dielectric constant, low dielectric loss tangent, high heat resistance, and low water absorption.

### CITATION LIST

### Patent Literature

Patent Literature 1: JP 2003-277440 A

### SUMMARY OF THE INVENTION

### Problems to be Solved by the Invention

An object of the present disclosure is to provide a compound which can be used for fabricating a cured product with dielectric properties which are suppressed from being deteriorated due to moisture absorption, a resin composition, a prepreg, a resin film, a metal-clad laminate, a printed wiring board, and a semiconductor package, which are formed using the compound.

### Means for Solving the Problem

Examples of embodiments will be listed below. The present invention is not limited to the following embodiments.

One embodiment relates to a compound including: an indene ring; a vinylbenzyl group; and an aromatic ring-containing group of 6 or more carbon atoms without a polymerizable carbon-carbon double bond, in which both of the vinylbenzyl group and the aromatic ring-containing group are directly bonded to the indene ring.

Another embodiment relates to a compound represented by formula (1).

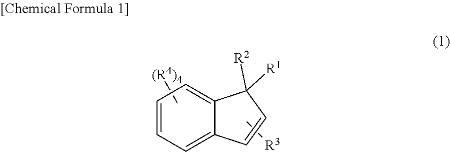

In formula (1), R¹ to R⁴ each independently represents a hydrogen atom or a monovalent substituent; at least one of R¹, R², or R³ is a vinylbenzyl group, and at least another one of R¹, R², or R³ is an aromatic ring-containing group of 6 or more carbon atoms without a polymerizable carbon-carbon double bond; and all four R⁴s may be different from each other, or two or more of four R⁴s may be identical to each other.

Another embodiment relates to a resin composition including: any one of the compounds.

Another embodiment relates to a prepreg including: the resin composition, or a semi-cured product of the resin composition.

Another embodiment relates to a resin film including: the resin composition, or a semi-cured product of the resin composition.

Another embodiment relates to a metal-clad laminate including: a cured product of the resin composition; and a metal foil.

Another embodiment relates to a printed wiring board including: a cured product of the resin composition.

Another embodiment relates to a semiconductor package including: the printed wiring board; and a semiconductor element.

Another embodiment relates to a semiconductor package including: a semiconductor element; and a cured product of the resin composition for sealing the semiconductor element.

Another embodiment relates to a printed wiring board including: at least one of a surface protective film or an interlayer insulating film formed with the resin composition.

### Advantageous Effects of the Invention

The present disclosure can provide a compound which can be used for fabricating a cured product with dielectric properties which are suppressed from being deteriorated due to moisture absorption, a resin composition, a prepreg, a resin film, a metal-clad laminate, a printed wiring board, and a semiconductor package, which are formed using the compound.

### BRIEF DESCRIPTION OF THE DRAWINGS

FIG. 1 illustrates an NMR spectrum of compound 1.

FIG. 2 illustrates an NMR spectrum of compound 2.

FIG. 3 illustrates an NMR spectrum of compound 3.

FIG. 4 illustrates an NMR spectrum of compound 4.

FIG. 5 illustrates an NMR spectrum of compound 5.

FIG. 6 illustrates an NMR spectrum of compound 6.

### MODES FOR CARRYING OUT THE INVENTION

Embodiments of the present invention will be described below in detail. The present invention is not limited to the following embodiments.

In the present disclosure, a numerical range indicated using “to” indicates a range including numerical values described before and after “to” as minimum and maximum values. In numerical ranges described in a stepwise manner in the present disclosure, an upper or lower limit of a certain numerical range may be replaced with an upper or lower limit of another numerical range. In addition, upper or lower limits of numerical ranges described in the present disclosure may be replaced with values shown in Examples.

In the present disclosure, each component may contain one or two or more substances corresponding thereto, unless otherwise specified.

In the present disclosure, when a plurality of substances corresponding to each component are present in a resin composition, the amount of each component in the resin composition means the total amount of the plurality of substances present in the resin composition, unless otherwise specified.

In the present disclosure, unless specifically described otherwise, the weight average molecular weight (Mw) and the number average molecular weight (Mn) are numerical values obtained through measurement by means of the following procedure.

The weight average molecular weight and the number average molecular weight are obtained through measurement by gel permeation chromatography (GPC), and conversion with the use of a calibration curve of standard polystyrene. The calibration curve is approximated by a cubic equation using standard polystyrene: TSKstandard POLYSTYRENE (Type; A-2500, A-5000, F-20, and F-80) (manufactured by Tosoh Corporation, product name). The conditions of GPC are as follows.
System: High Performance GPC System “HLC-8320GPC” (Tosoh Corporation, product name)
Detector: UV Detector “UV-8320” (Tosoh Corporation, product name)
Column: guard column: TSKgel guardcolumn Super (HZ)-M+, column: TSKgel
SuperMultipore HZ-M (two columns), reference column: TSKgel SuperH-RC (two columns) (all manufactured by Tosoh Corporation, product names)
Column sizes: 4.6 mm×20 mm (guard column), 4.6 mm×150 mm (column), 6.0 mm×150 mm (reference column)
Eluent: tetrahydrofuran
Sample concentration: 10 mg/1 mL
Injection volume: 20 μL or 2 μL
Flow rate: 0.35 mL/minute
Measurement temperature: 40° C.

### [Compound]

A compound of one embodiment of the present disclosure includes an indene ring, a vinylbenzyl group, and an aromatic ring-containing group of 6 or more carbon atoms without a polymerizable carbon-carbon double bond (hereinafter, the group may also be referred to as “aromatic ring-containing group X”), in which both of the vinylbenzyl group and the aromatic ring-containing group X are directly bonded to the indene ring. The compound may also be referred to as “compound A” below.

A vinylbenzyl group has a relatively low polarity, and therefore the group can contribute to excellent dielectric properties. Meanwhile, investigation by the present inventors has revealed that the dielectric properties of a cured product obtained using a compound with an indene ring and a vinylbenzyl group may deteriorate due to moisture absorption. The present inventors have found that, if an aromatic ring-containing group of 6 or more carbon atoms without a polymerizable carbon-carbon double bond is introduced into a compound together with an indene ring and a vinylbenzyl group, it is possible to suppress the deterioration in dielectric properties of a cured product obtained using the compound due to moisture absorption. Although not constrained by any particular theory, it is considered that this is because a water molecule is less likely to approach due to a bulky structure of the aromatic ring-containing group X, which is an aromatic ring-containing group of 6 or more carbon atoms.

In addition, it is also generally required that the coefficient of thermal expansion of a material used for an electronic device is not large, and the compound A can exhibit a favorable coefficient of thermal expansion. Although not constrained by any particular theory, it is speculated that the control of molecular motion by interactions between aromatic rings such as π-π interactions can contribute to the reduction in thermal expansion, for example.

The compound A can contain an indene ring, and one or more vinylbenzyl groups and one or more aromatic ring-containing groups X, both bonded to the indene ring.

Due to the compound A having one or more vinylbenzyl groups in one molecule, a cured product can be obtained by promoting the reaction of the vinylbenzyl groups by heating or the like.

The compound A may have two or more vinylbenzyl groups. The compound A may have two or more aromatic ring-containing groups X. The compound A may have one or two vinylbenzyl groups and one or two aromatic ring-containing groups X. The compound A may have a vinylbenzyl group and an aromatic ring-containing groups X such that the total number of the vinylbenzyl group and the aromatic ring-containing group X is two or three.

The compound A may be a hydrocarbon compound, for example.

The vinylbenzyl group of the compound A may further have a substituent. The vinylbenzyl group is, for example, represented by formula (2) below.

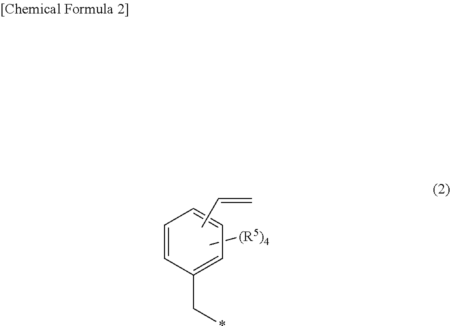

In formula (2), four R⁵s each independently represents a hydrogen atom or a monovalent substituent. The monovalent substituent represented by R⁵ may be a halogen atom or a monovalent organic group, for example. The monovalent organic group represented by R⁵ may be an alkyl group of 1 to 5 carbon atoms, an alkoxy group of 1 to 5 carbon atoms, a thioalkoxy group of 1 to 5 carbon atoms, or an aryl group of 6 to 20 carbon atoms, for example. The halogen atom may be a fluorine atom, a chlorine atom, a bromine atom, or an iodine atom. All of the four R⁵s may be different from each other, or two or more of the four R⁵s may be identical to each other. All of the four R⁵s may be hydrogen atoms, for example. In formula (2), a symbol * represents a bonding site to an indene ring.

The alkyl group of 1 to 5 carbon atoms represented by R⁵ may be a linear or branched alkyl group. Examples thereof include a methyl group, an ethyl group, a propyl group, an isopropyl group, an n-butyl group, an isobutyl group, a tert-butyl group, a sec-butyl group, a pentyl group, an isopentyl group, a neopentyl group, and the like.

The alkoxy group of 1 to 5 carbon atoms represented by R⁵ may be, for example, a group represented by R—O—, and R may be the alkyl group of 1 to 5 carbon atoms described above.

The thioalkoxy group of 1 to 5 carbon atoms represented by R⁵ may be, for example, a group represented by R—S—, and R may be the alkyl group of 1 to 5 carbon atoms described above.

The aryl group of 6 to 20 carbon atoms represented by R⁵ may be an atomic group in which one hydrogen atom has been removed from an aromatic hydrocarbon. Examples thereof include a phenyl group, a biphenyl-yl group, a terphenyl-yl group, a naphthalene-yl group, an anthracene-yl group, a tetracene-yl group, a fluorene-yl group, a phenanthrene-yl group, and derivatives thereof (however, a vinylbenzyl group and a derivative thereof are excluded).

The monovalent organic group represented by R⁵ may be a non-aromatic organic group, and may have 1 to 5 carbon atoms, 1 to 4 carbon atoms, 1 to 3 carbon atoms, or 1 to 2 carbon atoms. R⁵ may be a hydrogen atom, a halogen atom, an alkyl group of 1 to 5 carbon atoms, an alkoxy group of 1 to 5 carbon atoms, or a thioalkoxy group of 1 to 5 carbon atoms. Alternatively, R⁵ may be a hydrogen atom, a halogen atom, or an alkyl group of 1 to 5 carbon atoms. Preferably, R⁵ may be a hydrogen atom, a halogen atom, or an alkyl group of 1 to 5 carbon atoms. R⁵ may be a hydrogen atom, or an alkyl group having 1 to 5 carbon atoms, 1 to 4 carbon atoms, 1 to 3 carbon atoms, or 1 to 2 carbon atoms. Alternatively, R⁵ may be a hydrogen atom, a methyl group, or an ethyl group.

The vinylbenzyl group of the compound A may be any of an o-vinylbenzyl group, an m-vinylbenzyl group, and a p-vinylbenzyl group. When the compound A has two or more vinylbenzyl groups in one molecule, structural isomerism of the two or more vinylbenzyl groups may be the same or different from each other. From the viewpoint of dielectric properties, it is preferable that the vinylbenzyl compound has at least one p-vinylbenzyl group in one molecule.

In one molecule of the compound A, the amount of p-vinylbenzyl groups may be in a range from 10% by mass to 100% by mass, in a range from 20% by mass to 80% by mass, in a range from 30% by mass to 70% by mass, or in a range from 40% by mass to 60% by mass, relative to the total mass of all vinylbenzyl groups.

The vinylbenzyl group may be a monovalent group which can be directly bonded to a carbon atom of the 1-position, 2-position, or 3-position of the indene ring, for example. One vinylbenzyl group may be bonded to any one of carbon atoms of the 1-position, 2-position, or 3-position of the indene ring, for example. Two vinylbenzyl groups may be directly bonded to a carbon atom of the 1-position of the indene ring, for example. Two vinylbenzyl groups may be directly bonded to any two of carbon atoms of the 1-position, 2-position, or 3-position of the indene ring, respectively, for example. In all cases, the aromatic ring-containing group X can be further bonded to at least one of carbon atoms of substitutable position, from among carbon atoms of the 1-position, 2-position, and 3-position of the indene ring.

The 1-position to 7-position of the indene ring are as follows.

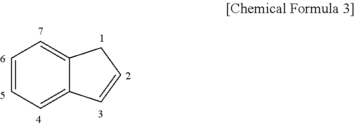

The aromatic ring-containing group (aromatic ring-containing group X) of 6 or more carbon atoms without a polymerizable carbon-carbon double bond may be a monovalent group. An aromatic ring in the aromatic ring-containing group X may be either a carbocyclic ring or a heterocyclic ring, but from the viewpoint of dielectric properties, the aromatic ring may be a carbocyclic ring. From the viewpoint of dielectric properties, it is preferable that the aromatic ring-containing group X is a monovalent hydrocarbon group containing a carbocyclic ring.

The aromatic ring-containing group X may contain any of a monocyclic ring, a condensed ring, or a polycyclic ring in which two or more rings selected from monocyclic rings and condensed rings are bonded via a single bond. However, from the viewpoint of curability, the number of rings is preferably three or less, and may be two or less or one. When the number of rings of the aromatic ring-containing group X is three or less, the reactivity of the vinylbenzyl group tends to be more effectively exhibited. The number of carbocyclic rings in the aromatic ring-containing group X is preferably three or less, and may be two or less or one, for example. The aromatic ring-containing group X may contain a benzene ring, a biphenyl structure, a terphenyl structure, a naphthalene ring, a fluorene ring, a phenanthrene ring, or the like, for example.

From the viewpoint of curability, the number of carbon atoms of the aromatic ring-containing group X may be 20 or less, 15 or less, 12 or less, or 10 or less. From the viewpoint of curability, the number of carbon atoms of the aromatic ring-containing group X may be 6 or more, 7 or more, or 8 or more. The number of carbon atoms of the aromatic ring-containing group X may be in a range from 6 to 20, in a range from 7 to 15, in a range from 8 to 12, or in a range from 8 to 10, for example.

The aromatic ring-containing group X may be an unsubstituted aryl group or an aryl group with a substituent without an a polymerizable carbon-carbon double bond, or an unsubstituted arylalkyl group or an arylalkyl group with a substituent without a polymerizable carbon-carbon double bond, for example. It is preferable that the aromatic ring-containing group X is an unsubstituted arylalkyl group or an arylalkyl group with a substituent without a polymerizable carbon-carbon double bond, from the viewpoint of being relatively similar to a vinylbenzyl group in which a vinyl group is bonded to a benzyl group, and thereby facilitating the synthesis of the compound A.

The aryl group is an atomic group in which one hydrogen atom has been removed from an aromatic hydrocarbon. Examples of the aryl group in the aromatic ring-containing group X include a phenyl group, a biphenyl-yl group, a terphenyl-yl group, a naphthalene-yl group, an anthracene-yl group, a tetracene-yl group, a fluorene-yl group, a phenanthrene-yl group, and the like.

The arylalkyl group is a group in which at least one of hydrogen atoms of an alkyl group has been substituted with an aryl group. Examples of the alkyl group of the arylalkyl group in the aromatic ring-containing group X include an alkyl group of 1 to 5 carbon atoms. More specific examples of the alkyl group include a methyl group, an ethyl group, an n-propyl group, an n-butyl group, an n-pentyl group, an isopropyl group, an isobutyl group, a sec-butyl group, a tert-butyl group, and the like. As the aryl group of the arylalkyl group in the aromatic ring-containing group X, the aryl group described above is exemplified. The number of carbon atoms of the unsubstituted monovalent arylalkyl group may be in a range from 7 to 20, in a range from 7 to 15, in a range from 8 to 12, or in a range from 8 to 10, for example. Examples of the arylalkyl group include a benzyl group, a phenethyl group, a naphthylmethyl group, a naphthylethyl group, and the like. It is preferable that the arylalkyl group is a benzyl group, from the viewpoint of being relatively similar to a vinylbenzyl group and thereby facilitating the synthesis of the compound A.

In the aromatic ring-containing group X, when the aryl group or the arylalkyl group has a substituent, it is preferable that the substituent does not have a carbon-carbon double bond. Examples of this kind of substituent include a halogen atom, a monovalent organic group without a carbon-carbon double bond, and the like. The halogen atom may be a fluorine atom, a chlorine atom, a bromine atom, or an iodine atom. The monovalent organic group without a carbon-carbon double bond may be an alkyl group of 1 to 5 carbon atoms, an alkoxy group of 1 to 5 carbon atoms, a thioalkoxy group of 1 to 5 carbon atoms, or an aryl group of 6 to 20 carbon atoms, for example. Examples of the alkyl group of 1 to 5 carbon atoms include a methyl group, an ethyl group, an n-propyl group, an n-butyl group, an n-pentyl group, an isopropyl group, an isobutyl group, a sec-butyl group, a tert-butyl group, and the like.

The aromatic ring-containing group X may be a group containing a heteroatom or a group containing no a heteroatom. The aromatic ring-containing group X may be a monovalent group without a heteroatom, for example.

Examples of the aromatic ring-containing group X include a methylbenzyl group, an ethylbenzyl group, a butylbenzyl group, a dimethylbenzyl group, a trimethylbenzyl group, and the like.

The aromatic ring-containing group X in the compound A may be a monovalent organic group represented by formula (3) below, for example.

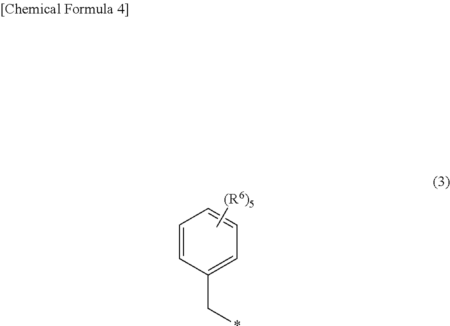

In formula (3), five R⁶s are each independently a hydrogen atom or a substituent. The substituent may be a halogen atom or a monovalent organic group, for example. The halogen atom and the monovalent organic group represented by R⁶ may be those represented by R⁵ in formula (2) above. All of the five R⁶s may be different from each other, or two or more of the five R⁶s may be identical to each other. All of the five R⁶s may be hydrogen atoms, for example. In another example, four of the five R⁶s may be hydrogen atoms, and the remaining one of the five R⁶s may be an alkyl group of 1 to 5 carbon atoms. Four of the five R⁶s may be hydrogen atoms, and the remaining one of the five R⁶s may be a methyl group or an ethyl group. Alternatively, four of the five R⁶s may be hydrogen atoms, and the remaining one of the five R⁶s may be a methyl group. When four of the five R⁶s are hydrogen atoms, and the remaining one of the five R⁶s is an alkyl group, the alkyl group may be bonded to any of the o-position, m-position, and p-position, but it is preferably that the alkyl group is bonded to the p-position.

The aromatic ring-containing group X may be an o-methylbenzyl group, an m-methylbenzyl group, or a p-methylbenzyl group, for example. The aromatic ring-containing group X may be a p-methylbenzyl group, for example.

When the compound A has two or more aromatic ring-containing groups X, the two or more aromatic ring-containing groups X may be the same or different from each other.

The aromatic ring-containing group X may be a monovalent group which can be directly bonded to a carbon atom of the 1-position, 2-position, or 3-position on the indene ring, for example. One aromatic ring-containing group X may be bonded to only one of carbon atoms of the 1-position, 2-position, or 3-position of the indene ring, for example. Two aromatic ring-containing groups X may be directly bonded to a carbon atom of the 1-position of the indene ring, for example. Two aromatic ring-containing groups X may be directly bonded to any two of carbon atoms of the 1-position, 2-position, or 3-position of the indene ring, respectively, for example. In all cases, a vinylbenzyl group may be further bonded to at least one of carbon atoms of substitutable position, from among carbon atoms of the 1-position, 2-position, and 3-position of the indene ring.

The compound A may further have a monovalent substituent other than the vinylbenzyl group and the aromatic ring-containing group X, at a carbon atom of an indene ring. The compound A may have a monovalent substituent other than the vinylbenzyl group and the aromatic ring-containing group X, at one or more of carbon atoms of the 4-position, 5-position, 6-position, or 7-position of the indene ring, for example.

Examples of the monovalent substituent other than the vinylbenzyl group and the aromatic ring-containing group X include a halogen atom, a monovalent organic group, and the like. The halogen atom may be a fluorine atom, a chlorine atom, a bromine atom, or an iodine atom. Examples of the monovalent organic group include an alkyl group of 1 to 5 carbon atoms, an alkoxy group of 1 to 5 carbon atoms, a thioalkoxy group of 1 to 5 carbon atoms, an aryl group of 6 to 20 carbon atoms, or the like. Examples of the alkyl group of 1 to 5 carbon atoms include a methyl group, an ethyl group, an n-propyl group, an n-butyl group, an n-pentyl group, an isopropyl group, an isobutyl group, a sec-butyl group, a tert-butyl group, and the like.

The compound A may be a compound having no monovalent substituent at carbon atoms of the 4-position, 5-position, 6-position, and 7-position of the indene ring, for example.

The compound A may be a compound having no monovalent substituent other than the vinylbenzyl group and the aromatic ring-containing group X at the carbon atoms of an indene ring, for example.

The compound A may have a structure in which one vinylbenzyl group is directly bonded to a carbon atom of the 1-position, 2-position, or 3-position of the indene ring, one aromatic ring-containing group X is directly bonded to a carbon atom of the 1-position, 2-position, or 3-position of the indene ring, and, of the carbon atoms constituting the indene ring, other carbon atoms have no substituent, for example. It is preferable that materials used for a metal-clad laminate, a prepreg, and the like used in an electronic device have favorable adhesive strength with a metal foil. If this kind of a structure is adopted, excellent adhesive strength with a metal foil is easily obtained.

It is possible to provide a low polymerization degree oligomer or prepolymer, in which a compound in which a vinylbenzyl group and an aromatic ring-containing group X has been introduced into an indene ring has been polymerized, using the compound A. It is preferable that both the oligomer and the prepolymer contain unreacted vinylbenzyl groups to some extent, and that a curing reaction starts by heating or the like.

Examples of the compound A include a compound represented by formula (1) below, for example.

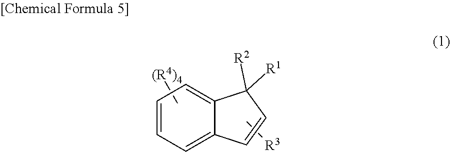

In formula (1), R¹ to R⁴ each independently represents a hydrogen atom or a monovalent substituent. At least one of R¹, R², or R³ is a vinylbenzyl group, and at least another one of R¹, R², or R³ is an aromatic ring-containing group X. All of the four R⁴s may be different from each other, or two or more of the four R⁴s may be identical to each other.

The vinylbenzyl group and the aromatic ring-containing group X are as described above.

The monovalent substituent represented by R¹, R², and R³ may be a halogen atom, a monovalent organic group, or the like. The halogen atom may be a fluorine atom, a chlorine atom, a bromine atom, or an iodine atom. The monovalent organic group may be an alkyl group of 1 to 5 carbon atoms, an alkoxy group of 1 to 5 carbon atoms, a thioalkoxy group of 1 to 5 carbon atoms, an aryl group of 6 to 20 carbon atoms, a vinylbenzyl group, or an aromatic ring-containing group X, for example. The alkyl group of 1 to 5 carbon atoms may be a methyl group, an ethyl group, an n-propyl group, an n-butyl group, an n-pentyl group, an isopropyl group, an isobutyl group, a sec-butyl group, or a tert-butyl group, for example.

A monovalent substituent represented by R⁴ may be a halogen atom, a monovalent organic group, or the like, for example. The halogen atom may be a fluorine atom, a chlorine atom, a bromine atom, or an iodine atom. The monovalent organic group may be an alkyl group of 1 to 5 carbon atoms, an alkoxy group of 1 to 5 carbon atoms, a thioalkoxy group of 1 to 5 carbon atoms, or an aryl group of 6 to 20 carbon atoms, for example. Examples of the alkyl group of 1 to 5 carbon atoms include those described above for R¹, R², and R³.

In formula (1), one or two of R¹, R², or R³ may be a vinylbenzyl group. Further, in formula (1), the remaining one or two of R¹, R², or R³ may be an aromatic ring-containing group X. In formula (1), one or two of R¹, R², or R³ may be a vinylbenzyl group, and the remaining one or two of R¹, R², or R³ may be an aromatic ring-containing group X. In formula (1), the total number of the vinylbenzyl group and the aromatic ring-containing group X may be two or three. In formula (1), all of four R⁴s may be hydrogen atoms.

From the viewpoint of adhesive strength with a metal foil, any one of R¹, R², or R³ may be a vinylbenzyl group, another one of R¹, R², or R³ may be an aromatic ring-containing group X, and still another one of R¹, R², or R³ may be a hydrogen atom, for example.

A method for synthesizing the compound A will be described. The compound A is specified by a molecular structure thereof regardless of the following synthesis method.

Examples of the method for synthesizing the compound A include a method including reacting a base compound having an indene ring, styrene having a methyl halide group, and a compound having an aromatic ring substituted with a halogen or an alkyl halide group, in the presence of a basic compound. Examples of the styrene having a methyl halide group include o-chloromethylstyrene, m-chloromethylstyrene, p-chloromethylstyrene, and the like. One of them may be used alone, or a mixture of two or more may be used. Examples of the compound having an aromatic ring substituted with a halogen or an alkyl halide group include a compound having an aromatic ring having a chloroalkyl group such as α-chloro-p-xylene. As the compound having an aromatic ring substituted with a halogen or an alkyl halide group, one compound may be used alone, or a mixture of two or more may be used. Examples of the basic compound include an alkali metal hydroxide, an alkali metal alkoxide, and the like.

A phase transfer catalyst may be used for performing the above reaction. Examples of the phase transfer catalyst include quaternary ammonium salts such as tetra-n-butylammonium chloride, tetra-n-butylammonium bromide, tetraethylammonium chloride, tetraethylammonium bromide, tetrapropylammonium chloride, tetrapropylammonium bromide, benzyltrimethylammonium chloride, benzyltrimethylammonium bromide, benzyltributylammonium chloride, benzyltributylammonium bromide, benzyldimethyltetradecylammonium chloride, tricaprylmethylammonium chloride, tetradecyltrimethylammonium bromide, hexadecyltrimethylammonium bromide, trioctylmethylammonium chloride, and tetra-n-butylammonium hydrogen sulfate; and quaternary phosphonium salts such as tetra-n-butylphosphonium chloride, tetra-n-butylphosphonium bromide, tetraphenylphosphonium chloride, tetraphenylphosphonium bromide, benzyltriphenylphosphonium chloride, and benzyltriphenylphosphonium bromide. The reaction can be performed by means of solution polymerization. The reaction may be performed under heating and stirring, for example. A polymerization inhibitor may be added to a reaction system. Examples of the polymerization inhibitor include hydroquinone, methylhydroquinone, tert-butylhydroquinone, 2,6-di-tert-butylhydroquinone, 2,5-di-tert-butylhydroquinone, hydroquinone monomethyl ether, 1,4-benzoquinone, 2-tert-butyl-1,4-benzoquinone, 2-tert-butylphenol, 2,4-di-tert-butylphenol, 2,6-di-tert-butylphenol, cresol, catechol, 4-tert-butylcatechol, pyrogallol, 4-methoxyphenol, thiodiphenylamine, phenothiazine, 3,7-dioctylphenothiazine, 3,7-dicumylphenothiazine, 2,2,6,6-tetramethylpiperidine-1-oxyl, 4-hydroxy-2,2,6,6-tetramethylpiperidine-1-oxyl, 4-benzoyloxy-2,2,6,6-tetramethylpiperidine-1-oxyl, and bis(2,2,6,6-tetramethyl-1-piperidinyloxy-4-yl) sebacate. The obtained product may be purified by means of a known method such as concentration, reprecipitation, or washing, if necessary.

A single compound may be obtained or a mixture of two or more compounds may be obtained.

A compound is synthesized in which a vinylbenzyl group is directly bonded to a carbon atom of at least one of the 1-position, 2-position, or 3-position of the indene ring, and an aromatic ring-containing group is directly bonded to a carbon atom of at least one of the 1-position, 2-position, or 3-position of the indene ring, for example. The mixture may contain two or more isomers with different bonding sites. Depending on synthetic conditions, the mixture may contain two or more compounds with different bonding numbers and bonding sites of the vinylbenzyl group and the aromatic ring-containing group X, to the indene ring. In addition to the compound A, the mixture may contain a compound in which only one of the vinylbenzyl group or the aromatic ring-containing group X is bonded to the indene ring, for example.

When a mixture of two or more compounds is obtained by the synthesis of the compound A, the weight average molecular weight (Mw) of the mixture is not particularly limited. However, from the viewpoint of moldability and handleability, the weight average molecular weight (Mw) of the mixture is preferably in a range from 240 to 350, more preferably in a range from 250 to 340, and even more preferably in a range from 260 to 330. The number average molecular weight (Mn) of the mixture is preferably in a range from 200 to 330, more preferably in a range from 210 to 320, and even more preferably in a range from 210 to 310.

The dielectric constant (Dk) of a cured product of the compound A at 25° C. and 10 GHz may be 4.0 or less, 3.5 or less, or 3.4 or less, for example. Meanwhile, the dielectric constant (Dk) of the cured product of the compound A at 25° C. and 10 GHz may be 2.3 or more, or 2.4 or more, for example. The dielectric constant (Dk) of the cured product of the compound A at 25° C. and 10 GHz may be in a range from 2.3 to 4.0, in a range from 2.3 to 3.5, or in a range from 2.4 to 3.4, for example. The dielectric loss tangent (Df) of the cured product of the compound A at 25° C. and 10 GHz may be 0.0020 or less, 0.0015 or less, 0.0013 or less, 0.0010 or less, or 0.0008 or less, for example. Meanwhile, the dielectric loss tangent (Df) of the cured product of the compound A at 25° C. and 10 GHz may be 0.0001 or more, or 0.0002 or more, for example. The dielectric loss tangent (Df) of the cured product of the compound A at 25° C. and 10 GHz may be in a range from 0.0001 to 0.0020, in a range from 0.0001 to 0.0015, in a range from 0.0001 to 0.0013, in a range from 0.0002 to 0.0010, or in a range from 0.0002 to 0.0008, for example.

The coefficient of thermal expansion of the cured product of the compound A at a temperature of 30° C. to 120° C. may be in a range from 30 ppm/° C. to 50 ppm/° C., for example.

When the mixture of two or more compounds is obtained by synthesis of the compound A, the dielectric constant (Dk) at 25° C. and 10 GHz, the dielectric loss tangent (Df) at 25° C. and 10 GHz, and/or the coefficient of thermal expansion at a temperature of 30° C. to 120° C. of the cured product of the mixture may be within the above ranges, for example.

A sample, of which the dielectric constant (Dk) at 25° C. and 10 GHz, the dielectric loss tangent (Df) at 25° C. and 10 GHz, and the coefficient of thermal expansion at a temperature of 30° C. to 120° C. of the cured product of the compound A are to be measured, is obtained by curing the compound A to be in a state of C-stage according to JIS K 6800 (1985). Specifically, the sample can be prepared by means of the method descried in the Examples.

The measurement of the dielectric constant (Dk) at 25° C. and 10 GHz and the dielectric loss tangent (Df) at 25° C. and 10 GHz may be performed according to the measurement of the dielectric constant (Dk) and the dielectric loss tangent (Df) at 25° C. and 10 GHz of a cured product of a resin composition, which will be described later. The measurement of the coefficient of thermal expansion at a temperature of 30° C. to 120° C. of the cured product of the compound A may be performed according to the measurement of the coefficient of thermal expansion at a temperature of 30° C. to 120° C. of a cured product of a resin composition, which will be described later.

The compound A may be used in a wide range of fields including mobile communication devices such as smartphones, base station devices thereof, network infrastructure devices such as servers, routers, and large servers, various electronic devices such as large computers, personal computers, and industrial computers, and electronic devices mounted in household appliances and automobiles.

### [Resin Composition]

A resin composition of one embodiment contains the compound A described above.

### <Compound A>

The resin composition may contain one compound A described alone or a combination of two or more compounds thereof.

From the viewpoint of dielectric properties and heat resistance, the amount of the compound A may be 10% by mass or more, 20% by mass or more, or 30% by mass or more, relative to the solid fraction amount of the resin composition. Meanwhile, from the viewpoint of dielectric properties and heat resistance, the amount of the compound A may be 100% by mass or less, 90% by mass or less, or 80% by mass or less, relative to the solid fraction amount of the resin composition. The amount of the compound A may be in a range from 10% by mass to 100% by mass, in a range from 20% by mass to 90% by mass, or in a range from 30% by mass to 80% by mass, relative to the solid fraction amount of the resin composition, for example.

In the present disclosure, a “solid fraction” of the resin composition refers to a component in the resin composition other than a volatile substance such as water or a solvent described later. The solid fraction is a component which remains without being volatilized when the resin composition is dried. The solid fraction may also include a liquid, starch syrup, or wax-like component at room temperature of about 25° C., and does not necessarily have to be a solid.

### <Other Components>

The resin composition may contain other components other than the compound A described above as necessary.

Examples of other components include thermosetting resins, fillers, elastomers, curing agents, curing accelerators, solvents, flame retardants, antioxidants, thermal stabilizers, antistatic agents, ultraviolet light absorbers, pigments, colorants, lubricants, and the like.

One of these other components may be used alone, or a combination of two or more may be used.

The resin composition may further contain a thermosetting resin in addition to the compound A described above.

As the thermosetting resin, a compound with a reactive group can be used. The thermosetting resin may be a compound with a reactive group, which can be self-cured or can be cured when a curing agent is used in combination, for example.

There are no particular limitations on the thermosetting resin, and examples of the thermosetting resin include an epoxy resin, a maleimide compound, a phenolic resin, a cyanate resin, an isocyanate resin, a benzoxazine resin, an oxetane resin, an amino resin, an unsaturated polyester resin, an allyl resin, a dicyclopentadiene resin, a silicone resin, a triazine resin, a melamine resin, and the like.

From thereamong, an epoxy resin, a phenolic resin, or a maleimide compound is preferable from the viewpoint of thermosetting properties and dielectric properties.

The epoxy resin preferably has two or more epoxy groups in one molecule. Examples of the epoxy resin include a glycidyl ether-type epoxy resin, a glycidyl amine-type epoxy resin, and a glycidyl ester-type epoxy resin. In each of the above types of epoxy resins, an epoxy resin can be further classified into a bisphenol type epoxy resin, an alicyclic epoxy resin, an aliphatic chain epoxy resin, a novolac type epoxy resin, a stilbene type epoxy resin, a naphthalene skeleton-containing epoxy resin, a biphenyl type epoxy resin, a xylylene type epoxy resin, and dihydroanthracene type epoxy resin.

Examples of the maleimide compound include a compound with one or more N-substituted maleimide groups and derivatives thereof. As the maleimide compound, it is possible to use at least one selected from the group consisting of a compound with two or more N-substituted maleimide groups and derivatives thereof, for example.

Examples of the compound with one or more N-substituted maleimide groups include an aromatic maleimide compound with an N-substituted maleimide group directly bonded to an aromatic ring, an aromatic bismaleimide compound with two N-substituted maleimide groups directly bonded to an aromatic ring, an aromatic polymaleimide compound with three or more N-substituted maleimide groups directly bonded to an aromatic ring, and an aliphatic maleimide compound with an N-substituted maleimide group directly bonded to an aliphatic hydrocarbon.

Specific examples of the compound with one or more N-substituted maleimide groups include N,N′-ethylenebismaleimide, N,N′-hexamethylenebismaleimide, N,N′-(1,3-phenylene)bismaleimide, N,N′-[1,3-(2-methylphenylene)]bismaleimide, N,N′-[1,3-(4-methylphenylene)]bismaleimide, N,N′-(1,4-phenylene)bismaleimide, bis(4-maleimidophenyl)methane, bis(3-methyl-4-maleimidophenyl)methane, 3,3′-dimethyl-5,5′-diethyl-4,4′-diphenylmethane bismaleimide, bis(4-maleimidophenyl)ether, bis(4-maleimidophenyl)sulfone, bis(4-maleimidophenyl)sulfide, bis(4-maleimidophenyl)ketone, bis(4-maleimidocyclohexyl)methane, 1,4-bis(4-maleimidophenyl)cyclohexane, 1,4-bis(maleimidomethyl)cyclohexane, 1,4-bis(maleimidomethyl)benzene, 1,3-bis(4-maleimidophenoxy)benzene, 1,3-bis(3-maleimidophenoxy)benzene, bis[4-(3-maleimidophenoxy)phenyl]methane, bis[4-(4-maleimidophenoxy)phenyl]methane, 1,1-bis[4-(3-maleimidophenoxy)phenyl]ethane, 1,1-bis[4-(4-maleimidophenoxy)phenyl]ethane, 1,2-bis[4-(3-maleimidophenoxy)phenyl]ethane, 1,2-bis[4-(4-maleimidophenoxy)phenyl]ethane, 2,2-bis[4-(3-maleimidophenoxy)phenyl]propane, 2,2-bis[4-(4-maleimidophenoxy)phenyl]propane, 2,2-bis[4-(3-maleimidophenoxy)phenyl]butane, 2,2-bis[4-(4-maleimidophenoxy)phenyl]butane, 2,2-bis[4-(3-maleimidophenoxy)phenyl]-1,1,1,3,3,3-hexafluoropropane, 2,2-bis[4-(4-maleimidophenoxy)phenyl]-1,1,1,3,3,3-hexafluoropropane, 4,4-bis(3-maleimidophenoxy)biphenyl, 4,4-bis(4-maleimidophenoxy)biphenyl, bis[4-(3-maleimidophenoxy)phenyl]ketone, bis[4-(4-maleimidophenoxy)phenyl]ketone, bis(4-maleimidophenyl)disulfide, bis[4-(3-maleimidophenoxy)phenyl]sulfide, bis[4-(4-maleimidophenoxy)phenyl]sulfide, bis[4-(3-maleimidophenoxy)phenyl]sulfoxide, bis[4-(4-maleimidophenoxy)phenyl]sulfoxide, bis[4-(3-maleimidophenoxy)phenyl]sulfone, bis[4-(4-maleimidophenoxy)phenyl]sulfone, bis[4-(3-maleimidophenoxy)phenyl]ether, bis[4-(4-maleimidophenoxy)phenyl]ether, 1,4-bis[4-(4-maleimidophenoxy)-α,α-dimethylbenzyl]benzene, 1,3-bis[4-(4-maleimidophenoxy)-α,α-dimethylbenzyl]benzene, 1,4-bis[4-(3-maleimidophenoxy)-α,α-dimethylbenzyl]benzene, 1,3-bis[4-(3-maleimidophenoxy)-α,α-dimethylbenzyl]benzene, 1,4-bis[4-(4-maleimidophenoxy)-3,5-dimethyl-α,α-dimethylbenzyl]benzene, 1,3-bis[4-(4-maleimidophenoxy)-3,5-dimethyl-α,α-dimethylbenzyl]benzene, 1,4-bis[4-(3-maleimidophenoxy)-3,5-dimethyl-α,α-dimethylbenzyl]benzene, 1,3-bis[4-(3-maleimidophenoxy)-3,5-dimethyl-α,α-dimethylbenzyl]benzene, polyphenylmethanemaleimide, an aromatic bismaleimide compound having an indane skeleton, and a biphenylaralkyl-type maleimide compound.

An example of a derivative of the compound with one or more N-substituted maleimide groups is an aminomaleimide compound having a structural unit derived from the compound with one or more N-substituted maleimide groups described above, and a structural unit derived from a diamine compound.

The amount of the compound A in the resin composition may be 10% by mass or more, 20% by mass or more, or 30% by mass or more, relative to the total amount of the thermosetting resin. Meanwhile, the amount of the compound A may be 100% by mass or less, 90% by mass or less, or 80% by mass or less, relative to the total amount of the thermosetting resin. The amount of the compound A may be in a range from 10% by mass to 100% by mass, in a range from 20% by mass to 90% by mass, or in a range from 30% by mass to 80% by mass, relative to the solid fraction amount of the resin composition, for example.

The amount of the thermosetting resin contained in the resin composition in addition to the compound A may be 50% by mass or more, 60% by mass or more, 70% by mass or more, or 100% by mass, relative to the total amount of the resin composition, for example.

The filler is preferably an inorganic filler, for example. Examples of the inorganic filler include silica (SiO₂), alumina (Al₂O₃), titanium oxide, barium titanate, strontium titanate, potassium titanate, calcium titanate, aluminum carbonate, magnesium hydroxide, aluminum hydroxide, aluminum silicate, calcium carbonate, calcium silicate, magnesium silicate, silicon nitride, boron nitride, aluminum borate, silicon carbide, mica, beryllia, clay, talc, and the like. From the viewpoint of dielectric properties, it is preferable that the inorganic filler is silica.

The shape and size of the filler are not particularly limited. The average particle size of the filler may be in a range from 0.01 μm to 20 μm, or in a range from 0.1 μm to 10 μm, for example. The average particle size of the filler is the particle size of a point corresponding to an integrated value of 50% in a volume-based particle distribution measured by means of a laser diffraction/scattering method.

Examples of the elastomer include a styrene-based elastomer, an olefin-based elastomer, a urethane-based elastomer, a polyester-based elastomer, a polyamide-based elastomer, an acrylic-based elastomer, a silicone-based elastomer, and the like.

As the curing accelerator, it is possible to use a radical polymerization initiator, for example. The radical polymerization initiator may be a thermal radical polymerization initiator or a photoradical polymerization initiator, but a thermal radical polymerization initiator is preferable. The radical polymerization initiator is not particularly limited, and examples thereof include an azo-based polymerization initiator, an organic peroxide-based polymerization initiator, and the like. Examples of the azo-based polymerization initiator include 2,2′-azobis(isobutyronitrile), 2,2′-azobis(2-methylpropanenitrile), 2,2′-azobis(2-methylbutyronitrile), 2,2′-azobis(4-methoxy-2,4-dimethylvaleronitrile), 2,2′-azobis(2-methylpropionic acid)dimethyl, 1,1′-azobis(methylcyclohexylcarboxylate), 2,2′-azobis[N-(2-propenyl)-2-methylpropionamide], 2,2′-azobis(N-butyl-2-methylpropionamide), 4,4′-azobis(3,3,4,4,5,5,6,6,7,7,8,8,8-tridecafluorooctyl 4-cyanopentanoate), 1,1′-azobis(cyclohexane-1-carbonitrile), and the like. Examples of the organic peroxide-based polymerization initiator include dicumyl peroxide, dibenzoyl peroxide, 2-butanone peroxide, tert-butyl perbenzoate, di-tert-butyl peroxide, 2,5-dimethyl-2,5-di(t-butyl peroxy) hexane, bis(tert-butyl peroxyisopropyl)benzene, tert-butyl hydroperoxide, and the like.

The resin composition may or may not contain a solvent. The solvent can further enhance coating properties by adjusting the viscosity of the resin composition. The solvent is preferably an organic solvent.

Examples of the organic solvent include alcohol-based solvents such as ethanol, propanol, butanol, methyl cellosolve, ethylene glycol monobutyl ether, and propylene glycol monomethyl ether; ketone-based solvents such as acetone, methylethylketone, methyl isobutyl ketone, and cyclohexanone; ether-based solvents such as tetrahydrofuran; aromatic hydrocarbon-based solvents such as toluene, xylene, and mesitylene; nitrogen atom-containing solvents such as dimethylformamide, dimethylacetamide, and N-methylpyrrolidone; a sulfur atom-containing solvent such as dimethylsulfoxide, and an ester-based solvent such as γ-butyrolactone.

When the resin composition contains a solvent, the solid fraction amount of the resin composition may be in a range from 30% by mass to 95% by mass, in a range from 40% by mass to 90% by mass, in a range from 50% by mass to 80% by mass, or in a range from 50% by mass to 70% by mass, relative to the total mass of the resin composition, for example.

### <Method for Producing Resin Composition>

A method for producing the resin composition is not particularly limited. The resin composition according to one embodiment is not limited by the method for producing the resin composition, and characteristics thereof are as described in the present disclosure. As an example of the method for producing the resin composition, the resin composition can be obtained using the compound A, and if necessary, adding an optional component to the compound A and mixing them. More specifically, for example, the resin composition can be obtained in such a manner that the compound A is dissolved or dispersed in a solvent, and, if necessary, other components are added thereto and they are mixed. Conditions such as the mixing order of each component, temperature, and time are not particularly limited, and conditions may be appropriately adjusted in accordance with the type of raw materials, the scale of production, a production apparatus, and the like.

### <Dielectric Properties of Cured Product of Resin Composition>

The dielectric constant (Dk) of a cured product of the resin composition at 25° C. and 10 GHz is preferably 4.0 or less, more preferably 3.5 or less, and even more preferably 3.4 or less. The smaller the dielectric constant (Dk) of the cured product of the resin composition at 25° C. and 10 GHz, the better, and there are no particular limitations on a lower limit thereof. However, the dielectric constant (Dk) may be 2.3 or more, or 2.4 or more, in consideration of the balance with other physical properties, for example.

The dielectric constant (Dk) of the cured product of the resin composition at 25° C. and 10 GHz may be in a range from 2.3 to 4.0, in a range from 2.3 to 3.5, or in a range from 2.4 to 3.4, for example.

The dielectric loss tangent (Df) of the cured product of the resin composition at 25° C. and 10 GHz is preferably 0.0020 or less, more preferably 0.0015 or less, even more preferably 0.0013 or less, even more preferably 0.0010 or less, and even more preferably 0.0008 or less. The smaller the dielectric loss tangent (Df) of the cured product of the resin composition at 25° C. and 10 GHz, the better, and there are no particular limitations on a lower limit thereof. However, the dielectric loss tangent (Df) may be 0.0001 or more, or 0.0002 or more in consideration of balance with other physical properties, for example.

The dielectric loss tangent (Df) of the cured product of the resin composition at 25° C. and 10 GHz may be in a range from 0.0001 to 0.0020, in a range from 0.0001 to 0.0015, in a range from 0.0001 to 0.0013, in a range from 0.0002 to 0.0010, or in a range from 0.0002 to 0.0008, for example.

In the present disclosure, the dielectric constant (Dk) and the dielectric loss tangent (Df) at 25° C. and 10 GHz are measured at 25° C. in 10 GHz bands in accordance with an SPDR method (split post dielectric resonator). For measuring devices, it is possible to use both a vector network analyzer E8364B, manufactured by Agilent Technologies, Inc. and a 10 GHz SPDR, manufactured by Vega Technology, Inc.

A sample, of which the dielectric constant (Dk) and the dielectric loss tangent (Df) of the cured product of the resin composition are to be measured, is obtained by curing the resin composition to be in a C-stage state according to JIS K 6800 (1985). Specifically, the sample can be prepared by means of the method described in the Examples.

### <Coefficient of Thermal Expansion of Cured Product of Resin Composition>

The coefficient of thermal expansion of the cured product of the resin composition at a temperature of 30° C. to 120° C. may be in a range from 30 ppm/° C. to 50 ppm/° C., for example. In the present disclosure, the coefficient of thermal expansion at a temperature of 30° C. to 120° C. is measured in accordance with the IPC (The Institute for interconnecting and Packaging Electronic Circuits) standard. For a measuring device, it is possible to use a thermomechanical analyzer (TMA) (manufactured by TA Instruments Japan Inc., product name: Q400). A sample to be measured is obtained by curing the resin composition to be in a C-stage state according to JIS K 6800 (1985). Specifically, the sample can be prepared by means of the method described in the Examples.

### [Prepreg]

According to an embodiment, it is possible to provide a prepreg containing a resin composition or a semi-cured product of the resin composition. The prepreg can be formed using a resin composition and a fibrous substrate, for example. As the resin composition, it is possible to use the resin composition described above. The details of the resin composition are as described above.

The prepreg can contain the resin composition described above, or the semi-cured product of the resin composition described above.

In the present disclosure, an example of an index of the semi-cured product is a B-stage state in JIS K 6800 (1985).

The prepreg may contain the resin composition or the semi-cured product of the resin composition, and further a fibrous substrate such as a sheet-like fibrous substrate, for example. In the prepreg, the resin composition may be uncured, or the resin composition may be partially or entirely semi-cured.

The prepreg can be obtained by coating a resin composition on a fibrous substrate and drying, for example. The prepreg can be obtained by coating a resin composition on a fibrous substrate by impregnation, and drying the fibrous substrate impregnated with the resin composition, for example. The drying is preferably performed at or above a temperature at which volatile components such as solvents that may be contained in the resin composition are removed. The drying may be performed at or above a temperature at which a thermosetting resin contained in the resin composition is semi-cured, depending on the application. The drying is preferably controlled such that a thermosetting resin contained in the resin composition is not completely cured. From this viewpoint, the drying temperature may be in a range from 80° C. to 200° C., for example. The drying time may be in a range from 1 minute to 30 minutes depending on the drying temperature, a drying apparatus, and a scale thereof, for example.

The fibrous substrate may be a woven fabric, a knitted fabric, or a nonwoven fabric. The fibrous substrate may be provided in the form of a chopped strand mat, a roving, or the like.

The fiber material may be either an inorganic fiber or an organic fiber.

Examples of the inorganic fiber include a glass fiber, a carbon fiber, and the like. Examples of the glass fiber include E-glass, NE-glass, D-glass, S-glass, Q-glass, and the like. Examples of the organic fiber include polyimide, polyester, tetrafluoroethylene, and the like. In the fibrous substrate, only one type of these fibers may be used or a combination of two or more types of these fibers may be used.

The material of the fibrous substrate is preferably an inorganic fiber and more preferably a glass fiber, from the viewpoint of dielectric properties and heat resistance.

The fibrous substrate may be appropriately selected depending on the use application of the prepreg, and preferably a sheet-like fibrous substrate.

The sheet-like fibrous substrate may be any of various sheet-like fibrous substrates used in known laminates for electrically insulating materials, for example.

The thickness of the sheet-like fibrous substrate is not particularly limited, but is preferably in a range from 0.02 mm to 0.5 mm, for example. The thicknesses at five points at equal distances on an entire surface of the sheet-like fibrous substrate are measured, and an arithmetic mean value of the five points refers to the thickness above.

### [Resin Film]

According to an embodiment, it is possible to provide a resin film containing a resin composition or a semi-cured product of the resin composition. The details of the resin composition are as described above.

The resin film may contain the resin composition described above, or the semi-cured product of the resin composition described above. In the resin film, the resin composition may be uncured, or the resin composition may be partially or entirely semi-cured. A cured product can be obtained by performing a heat treatment or the like to the resin film to cure the film, for example.

The resin film can be obtained by coating a resin composition on a coating target material and drying, for example. The drying may be performed in the same manner as the method for producing the prepreg, for example. After the resin film is dried on the coating target material, a product may be provided as a combination of the resin film and the coating target material. In this method, it is possible to provide a resin film for forming an insulating layer or the like on a coating target material in an electronic device or the like, for example. In another method, after a resin film is dried on a coating target material, the resin film is peeled off from the coating target material to provide the resin film as a product.

The coating target material may be either an inorganic substrate or an organic substrate. Examples of the coating target material include a glass substrate, a metal substrate such as a metal foil or a metal plate, a plastic substrate such as a plastic plate or a plastic film, a paper substrate, and the like. The coating target material may be the fibrous substrate described for the prepreg above. In order to peel off the resin film from the coating target material and provide the resin film as a product, it is possible to use a coating target material having a release layer formed on a surface thereof.

### [Metal-Clad Laminate]

According to one embodiment, it is possible to provide a metal-clad laminate containing a cured product of a resin composition, and a metal foil. The metal-clad laminate may contain a cured product of a prepreg and a metal foil, for example.

According to another embodiment, it is possible to provide a metal-clad laminate formed using a prepreg and a metal foil. The metal-clad laminate may contain a cured product of a prepreg and a metal foil, for example.

The details of the resin composition and the prepreg are as described above. In the present disclosure, an example of an index of the cured product is a C-stage state in JIS K 6800 (1985).

It is preferable that a metal-clad laminate includes a cured resin layer containing a cured resin product and a metal foil disposed on at least one surface of the cured resin layer. The cured resin layer contains the cured product of the resin composition described above. The cured resin layer may be a cured product of the prepreg described above. In the metal-clad laminate, for example, a metal foil is disposed on at least one surface of the cured product of the prepreg. It is more preferable that a metal foil is disposed on both surfaces of the cured product of the prepreg. The metal-clad laminate may be produced such that a metal foil is disposed on at least one surface of one sheet-like prepreg. Alternatively, the metal-clad laminate may be produced such that two or more sheet-like prepregs are laminated and a metal foil is disposed on at least one outermost surface of the laminate. The metal-clad laminate may be produced such that two or more sheet-like prepregs are laminated and a metal foil is disposed on both surfaces of this laminate, for example.

As a specific example of a method for producing a metal-clad laminate, a method in which a metal foil is disposed on a laminate of two or more sheet-like prepregs will be described below.

First, two or more sheet-like prepregs are laminated to obtain a laminate. The two or more sheet-like prepregs in the laminate may be identical to each other, or partially or entirely different from each other. It is sufficient if at least one of the two or more sheet-like prepregs in the laminate is obtained using the resin composition according to an embodiment.

Next, a metal foil is disposed on at least one surface of this laminate.

The laminate on which the metal foil is disposed is heated, and pressure is applied thereto. This promotes a curing reaction of the sheet-like prepregs, and accordingly it is possible to obtain cured products of the prepregs. Further, sheet-like prepregs adjacent to each other can be fixed. Heating and pressure conditions are not particularly limited, but a temperature may be in a range from 100° C. to 300° C., a time may be in a range from 10 minutes to 300 minutes, and a pressure may be in a range from 1.5 MPa to 5 MPa, for example. After heating and applying pressure to the laminate, the laminate may be reheated in order to further proceed the curing of the prepregs. In this case, a reheating temperature may be in a range from 100° C. to 300° C.

For performing pressure application, it is possible to use an autoclave molding machine, a multistage press, a multistage vacuum press, a continuous molding machine, or the like, for example.

A metal of the metal foil is not particularly limited, and examples thereof include copper, nickel, aluminum, gold, silver, platinum, molybdenum, ruthenium, tungsten, iron, titanium, chromium, and an alloy containing two or more metal elements from thereamong. Industrially, it is preferable that the metal is a simple metal such as copper, nickel, or aluminum. If copper is used as the metal foil, it is possible to provide a copper-clad laminate.

The dielectric properties of an evaluation piece obtained by removing a metal foil from a metal-clad laminate (hereinafter, also referred to as “metal foil removed evaluation piece”) are preferably as follows.

The dielectric constant (Dk) of the metal foil removed evaluation piece at 25° C. and 10 GHz is preferably 4.0 or less, more preferably 3.5 or less, and even more preferably 3.4 or less. The smaller the dielectric constant (Dk) of the metal foil removed evaluation piece at 25° C. and 10 GHz, the better, and there are no particular limitations on a lower limit thereof. However, the dielectric constant (Dk) may be 2.3 or more, or 2.4 or more, in consideration of the balance with other physical properties, for example.

The dielectric constant (Dk) of the metal foil removed evaluation piece at 25° C. and 10 GHz may be in a range from 2.3 to 4.0, in a range from 2.3 to 3.5, or preferably in a range from 2.4 to 3.4, for example.

The dielectric loss tangent (Df) of the metal foil removed evaluation piece at 25° C. and 10 GHz is preferably 0.0020 or less, more preferably 0.0015 or less, and even more preferably 0.0013 or less. The smaller the dielectric loss tangent (Df) of the metal foil removed evaluation piece at 25° C. and 10 GHz, the better, and there are no particular limitations on a lower limit thereof. However, the dielectric loss tangent (Df) may be 0.0001 or more, 0.0002 or more, or 0.0005 or more, in consideration of the balance with other physical properties, for example.

The dielectric loss tangent (Df) of the metal foil removed evaluation piece at 25° C. and 10 GHz may be in a range from 0.0001 to 0.0020, in a range from 0.0002 to 0.0015, or in a range from 0.0005 to 0.0013, for example.

The measurement of the dielectric constant (Dk) and the dielectric loss tangent (Df) of the metal foil removed evaluation piece at 25° C. and 10 GHz may be performed according to the measurement of the dielectric constant (Dk) and the dielectric loss tangent (Df) of the cured product of the resin composition at 25° C. and 10 GHz.

The coefficient of thermal expansion of the metal foil removed evaluation piece at a temperature of 30° C. to 120° C. (also simply referred to as “the coefficient of thermal expansion of the metal-clad laminate at a temperature of 30° C. to 120° C.”) may be in a range from 30 ppm/° C. to 50 ppm/° C., for example. The measurement of the coefficient of thermal expansion of the metal foil removed evaluation piece at a temperature of 30° C. to 120° C. may be performed according to the measurement of the coefficient of thermal expansion of the cured product of the resin composition at a temperature of 30° C. to 120° C.

### [Printed Wiring Board]

According to an embodiment, it is possible to provide a printed wiring board containing a cured product of a resin composition. According to an embodiment, it is possible to provide a printed wiring board containing at least one of a surface protective film or an interlayer insulating film formed with the resin composition. The details of the resin composition are as described above.

The cured product of the resin composition in the printed wiring board can be produced using a resin composition, a prepreg, a resin film, a metal-clad laminate, or a combination thereof. It is possible to provide a printed wiring board by forming wiring on a cured product of a prepreg by means of a known method, for example. In another example, it is possible to provide a printed wiring board using a metal-clad laminate and forming wiring by means of a known method. The printed wiring board may be a combination of these examples. The details of the prepreg, the resin film, and the metal-clad laminate are as described above.

The printed wiring board may be either a single-layer printed wiring board or a multi-layer printed wiring board.

### [Semiconductor Package]

According to one embodiment, it is possible to provide a semiconductor package including a printed wiring board and a semiconductor element. More specifically, it is possible to provide a semiconductor package including a semiconductor element, and a printed wiring board containing a cured product of a resin composition, for example. The details of the resin composition are as described above. The details of the cured product of the resin composition, and the printed wiring board are also as described above. The semiconductor package can be produced by mounting a semiconductor element, a memory, and the like on a printed wiring board by means of a known method, for example. The cured product of the resin composition may be used as an insulating material, a sealing material, or the like of the semiconductor package. It is possible to provide a semiconductor package including a semiconductor element, and a cured product of a resin composition for sealing the semiconductor element, for example.

Preferred examples of the embodiments of the present disclosure will be described below. The embodiments of the present invention are not limited to the following examples.

<1>

A compound including: an indene ring; a vinylbenzyl group; and an aromatic ring-containing group of 6 or more carbon atoms without a polymerizable carbon-carbon double bond, in which both of the vinylbenzyl group and the aromatic ring-containing group are directly bonded to the indene ring.

<2>

A compound represented by formula (1):

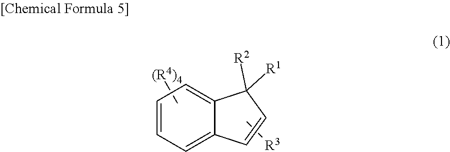

where in formula (1), R¹ to R⁴ each independently represents a hydrogen atom or a monovalent substituent; at least one of R¹, R², or R³ is a vinylbenzyl group, and at least another one of R¹, R², or R³ is an aromatic ring-containing group of 6 or more carbon atoms without a polymerizable carbon-carbon double bond; and all four R⁴s may be different from each other, or two or more of four R⁴s may be identical to each other.
   <3>

The compound according to <1> or <2>, in which the aromatic ring-containing group is an unsubstituted aryl group or an aryl group with a substituent without a polymerizable carbon-carbon double bond, or an unsubstituted arylalkyl group or an arylalkyl group with a substituent without a polymerizable carbon-carbon double bond.

<4>

The compound according to <3>, in which the aromatic ring-containing group is an unsubstituted benzyl group or a benzyl group with a substituent without a polymerizable carbon-carbon double bond.

<5>

A resin composition including: the compound according to any one of <1> to <4>.

<6>

A prepreg including: the resin composition according to <5>, or a semi-cured product of the resin composition.

<7>

A resin film including: the resin composition according to <5>, or a semi-cured product of the resin composition.

<8>

A metal-clad laminate including: a cured product of the resin composition according to <5>; and a metal foil.

<9>

A printed wiring board including: a cured product of the resin composition according to <5>.

<10>

A semiconductor package including: the printed wiring board according to <9>; and a semiconductor element.

<11>

A semiconductor package including: a semiconductor element; and a cured product of the resin composition according to <5> for sealing the semiconductor element.

<12>

A printed wiring board including: at least one of a surface protective film or an interlayer insulating film formed with the resin composition according to <5>.

The disclosure of the present application is related to the subject matter described in Japanese Patent Application No. 2023-099182 filed on Jun. 16, 2023, the entire disclosed contents of which are incorporated herein by reference.

### EXAMPLES

Hereinafter, the present invention will be described more specifically by way of examples, but the present invention is not limited to the examples below.

### <Method for Measuring Weight Average Molecular Weight (Mw) and Number Average Molecular Weight (Mn)>

The weight average molecular weight and number average molecular weight were obtained through measurement by gel permeation chromatography (GPC), and conversion with the use of a calibration curve of standard polystyrene. The calibration curve was approximated by a cubic equation using standard polystyrene: TSKstandard POLYSTYRENE (Types A-2500, A-5000, F-20, and F-80) (manufactured by Tosoh Corporation, product name). The measurement conditions of GPC are as described below.
System: High Performance GPC System HLC-8320GPC (Tosoh Corporation, product name)
Detector: UV Detector UV-8320 (Tosoh Corporation, product name)
Column: guard column: TSKgel guardcolumn Super (HZ)-M+, column: TSKgel SuperMultipore HZ-M (two columns), reference column: TSKgel SuperH-RC (two columns) (all manufactured by Tosoh Corporation, product names)
Column sizes: 4.6 mm×20 mm (guard column), 4.6 mm×150 mm (column), 6.0 mm×150 mm (reference column)
Eluent: tetrahydrofuran
Sample concentration: 10 mg/l mL
Injection volume: 20 μL or 2 μL
Flow rate: 0.35 mL/minute
Measurement temperature: 40° C.

### <Production of Compound>

The following were added to a reaction container with a volume of 500 mL, equipped with a stirrer, a thermometer, a reflux tube, and a nitrogen blowing port: indene, following chloromethylstyrene (CMS), α-chloro-p-xylene (CPX), tetra-n-butylammonium bromide (manufactured by KANTO CHEMICAL CO., INC.) as a phase transfer catalyst, phenothiazine as a polymerization inhibitor, and toluene as a solvent in the amounts shown in Table 1. The mixture was heated and stirred at 40° C., while blowing nitrogen thereto at a flow rate of 50 mL/minute.

Chloromethylstyrene “CMS-P”: AGC Seimi Chemical Co., Ltd., a mixture of m-isomer and p-isomer, the m-isomer amount is 50% by mass, and the p-isomer amount is 50% by mass

Next, the following aqueous solution of sodium hydroxide in the amount shown in Table 1 was added dropwise for 20 minutes, and the mixture was further stirred at 60° C. for nine hours. Blowing of nitrogen was continued during the reaction. The mixture was cooled to room temperature (25° C.), and neutralized with a 10% aqueous solution of hydrochloric acid. Thereafter, the mixture was washed twice with pure water, and toluene was removed by distillation under reduced pressure. Thereafter, a viscous liquid obtained was washed with methanol and then dried in vacuum. Accordingly, compounds 1 to 7 were obtained.

Aqueous solution of sodium hydroxide: an aqueous solution of sodium hydroxide with a concentration of 48% by mass, KANTO CHEMICAL CO., INC.

TABLE 1

| (Unit: parts by mass) | Production examples | | | | | | |
|---|---|---|---|---|---|---|---|
| Compound number | Compound 1 | Compound 2 | Compound 3 | Compound 4 | Compound 5 | Compound 6 | Compound 7 |
| Indene | 35.6 | 35.6 | 35.6 | 35.6 | 35.6 | 35.6 | 35.6 |
| CMS | 59.5 | 82.6 | 101.2 | 68.7 | 75.5 | 59.5 | 101.2 |
| CPX | 33.1 | 16.6 | 24.1 | 29.0 | 22.9 | 53.2 | 0 |
| Tetra-n-butylammonium | 7.1 | 7.1 | 9.1 | 7.1 | 7.1 | 8.4 | 7.1 |
| bromide | | | | | | | |
| Phenothiazine | 0.1 | 0.1 | 0.1 | 0.1 | 0.1 | 0.1 | 0.1 |
| Toluene | 73.0 | 76.5 | 91.6 | 75.7 | 76.1 | 84.4 | 77.6 |
| Aqueous solution of | 97.0 | 97.0 | 122.9 | 97.0 | 97.0 | 114.1 | 97.0 |
| sodium hydroxide | | | | | | | |

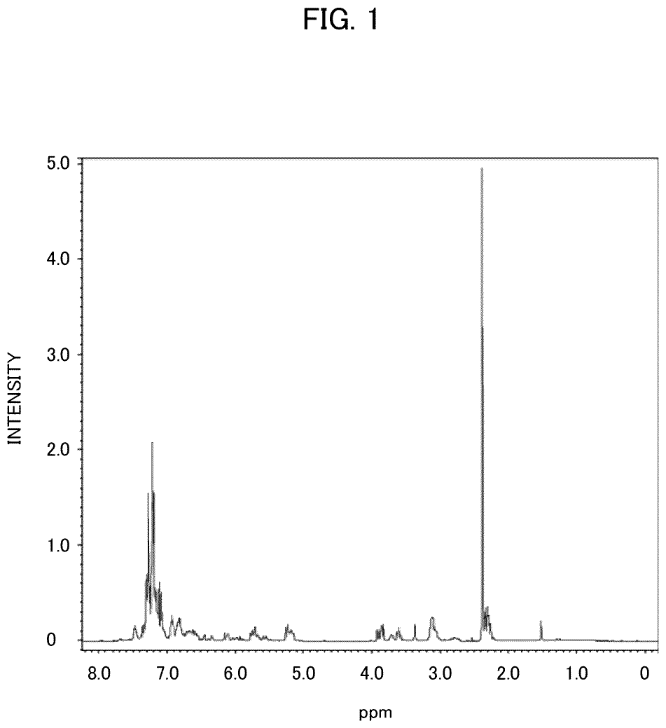

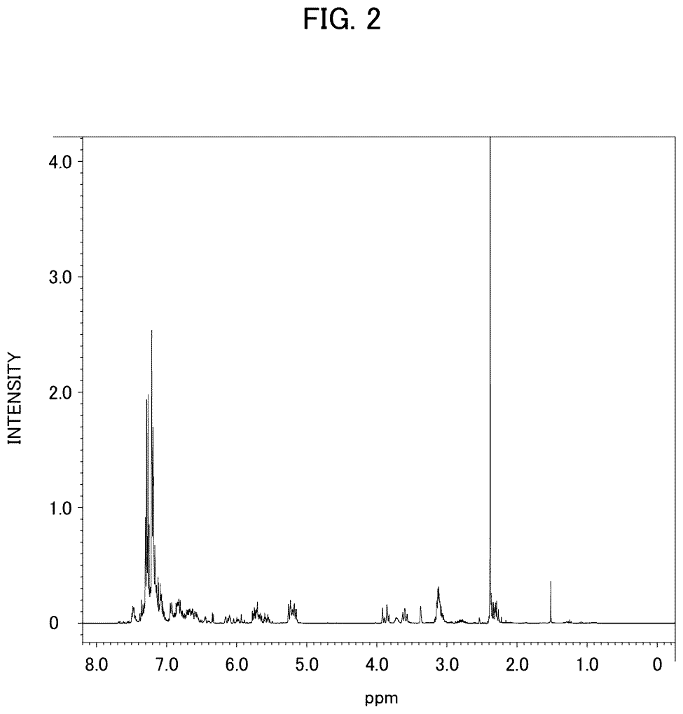

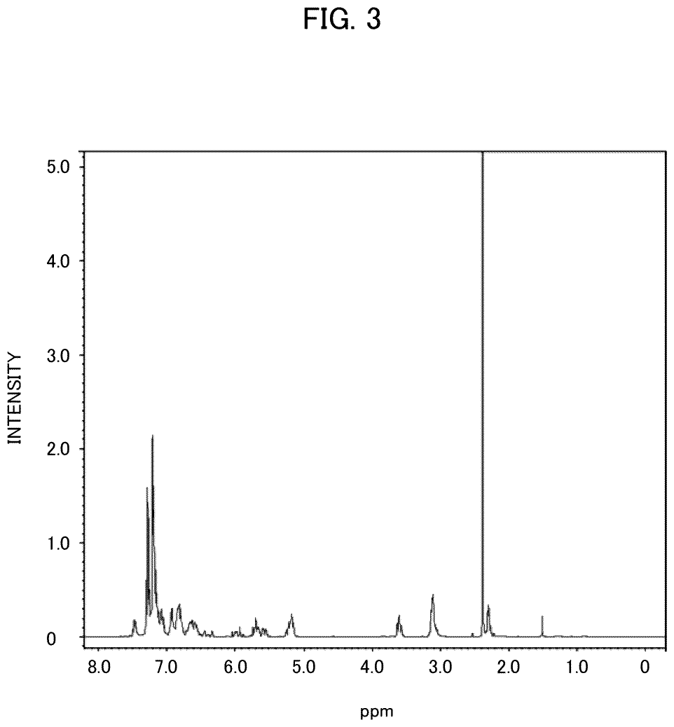

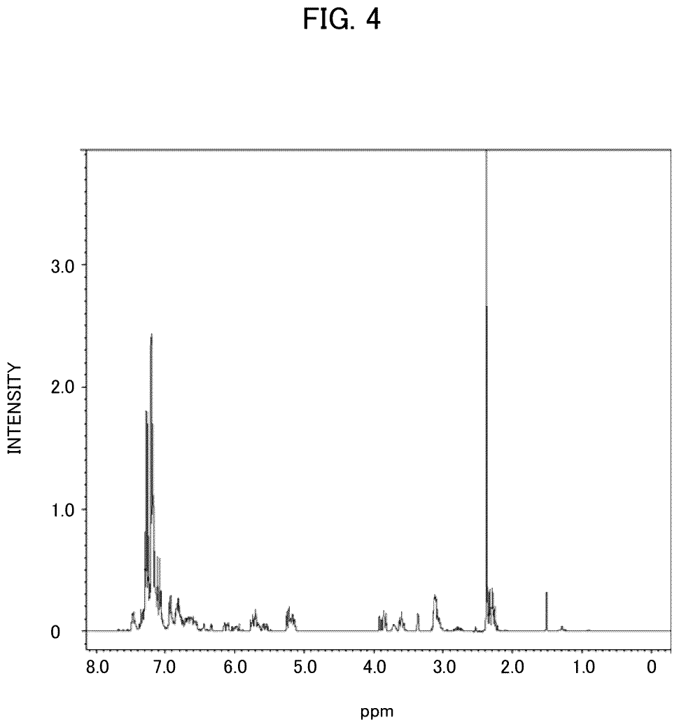

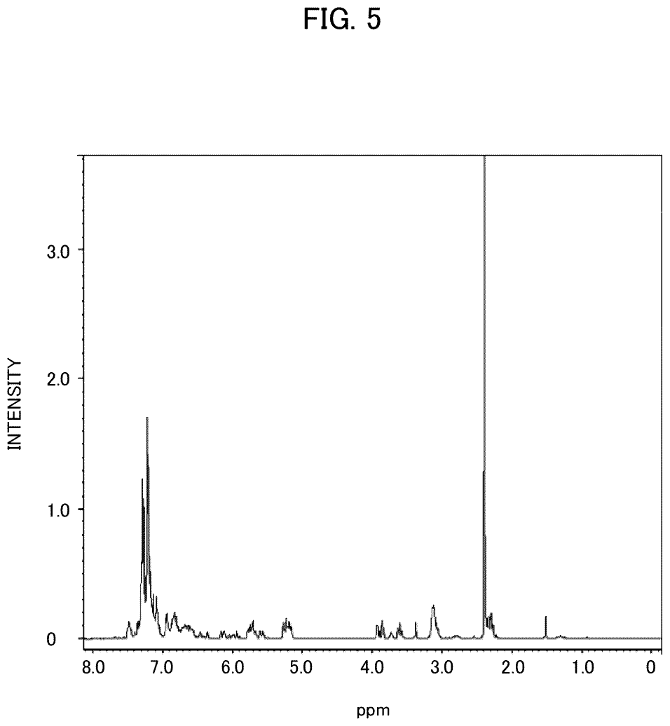

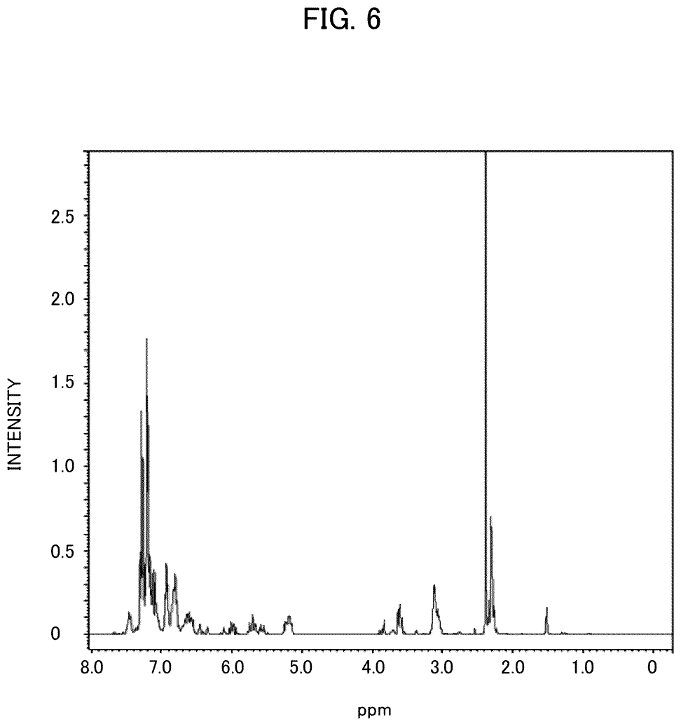

¹H-NMR spectra (0.2 Hz, CDCl₃, reference substance: tetramethylsilane (TMS)) were measured using ECX400II manufactured by JEOL Ltd. The ¹H-NMR analysis revealed that reactions in which a vinylbenzyl group is bonded to any of the 1-position, 2-position, or 3-position of the indene ring, and a methylbenzyl group is bonded to any of the 1-position, 2-position, or 3-position of the indene ring occurred, from the shift or disappearance of a peak of a raw material, specifically, the shift or disappearance of a peak of H of the 1-position of indene, the shift or disappearance of a peak of H of the 2-position or 3-position of indene, the shift of methylene peaks of CMS and CPX, the shift of a peak of vinyl of CMS, and the shift of a peak of methyl of CPX. FIGS. 1 to 6 illustrate the 1H-NMR spectra of compounds 1 to 6.

Further, GPC analysis revealed that the compounds 1 to 6 were the mixture of a compound into which one substituent selected from the group consisting of a vinylbenzyl group and a methylbenzyl group is introduced (mono-substituted compound), a compound into which two substituents are introduced (di-substituted compound), and a compound into which three substituents are introduced (tri-substituted compound).

Table 2 shows the yield (%), the ratio of the tri-substituted compound, di-substituted compound, and mono-substituted compound by GPC (intensity ratio), the weight average molecular weight (Mw), and the number average molecular weight (Mn) of the obtained compounds 1 to 7.

TABLE 2

| | Production examples | | | | | | |
|---|---|---|---|---|---|---|---|
| Compound number | Compound 7 | Compound 1 | Compound 2 | Compound 3 | Compound 4 | Compound 5 | Compound 6 |
| Yield (%) | 85.1 | 84.5 | 78.7 | 82.6 | 88.6 | 87.2 | 89.8 |
| Tri-substituted | 48.7 | 46.0 | 51.2 | 78.1 | 50.3 | 50.5 | 70.8 |
| compound | | | | | | | |
| (intensity ratio) | | | | | | | |
| Di-substituted | 40.5 | 42.8 | 39.2 | 21.0 | 40.1 | 39.4 | 27.2 |
| compound | | | | | | | |
| (intensity ratio) | | | | | | | |
| Mono-substituted | 10.8 | 11.2 | 9.6 | 0.9 | 9.6 | 10.1 | 2.1 |
| compound | | | | | | | |
| (intensity ratio) | | | | | | | |
| Mn/Mw | 238/287 | 217/264 | 229/277 | 302/323 | 225/271 | 226/273 | 277/304 |

### Examples 1 to 6 and Comparative Example 1

The compounds 1 to 7 obtained above were used in Examples 1 to 6 and Comparative Example 1. Specifically, as shown in Table 3, compound 7 was used in Comparative Example 1, compound 1 was used in Example 1, compound 4 was used in Example 2, compound 5 was used in Example 3, compound 2 was used in Example 4, compound 6 was used in Example 5, and compound 3 was used in Example 6.

### Resin Compositions of Examples 7 to 9 and Comparative Example 2

Resin compositions of Examples 7 to 9 and Comparative Example 2, having a solid content concentration of about 60% by mass, were prepared by mixing and stirring the components shown in Table 4 with toluene at 25° C. in accordance with the blend amounts shown in Table 4. In Table 1, when a component is a solution, a unit is parts by mass in terms of the solid fraction equivalent.

The details of each material shown in Tables 3 and 4 are as follows.
Compounds 1 to 7: Produced as Above
Maleimide compound: an aromatic maleimide compound having an indane skeleton represented by formula below

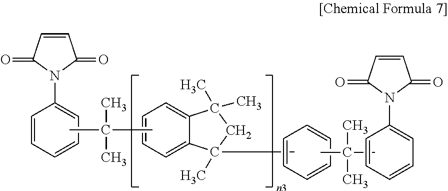

(In the above formula, n is a number in a range from 0.95 to 10.0.)

### <Method for Fabricating Cured Product>

### Examples 1 to 6 and Comparative Example 1

For Comparative Example 1 and Examples 1 to 6, a double-side copper foil-attached resin plates was obtained as follows.

On a low-profile copper foil with a thickness of 18 μm (manufactured by MITSUI MINING & SMELTING CO., LTD., product name: SI-VSP-18), a Teflon (registered trademark) sheet which was die-cut to have a size of 0.8 mm in thickness×70 mm in length×50 mm in width was arranged, and the powders of the compounds shown in Table 3 (compounds 1 to 7) were introduced into the die-cut portion. Next, a low-profile copper foil with a thickness of 18 μm (manufactured by MITSUI MINING & SMELTING CO., LTD., product name: SI-VSP-18) was also arranged on the side where the compound powders were introduced of the Teflon (registered trademark) sheet. The two copper foils were arranged such that M surfaces (matte surfaces) were in contact with the introduced compound powders. Subsequently, curing was performed by heating, applying pressure thereto, and molding in a vacuum state under conditions where a temperature was 230° C., a pressure was 2.0 MPa, and a time was 60 minutes, to obtain the double-side copper foil-attached resin plate.

### Examples 7 to 9 and Comparative Example 2

For Comparative Example 2 and Examples 7 to 9, a double-side copper foil-attached resin plate was obtained as follows. Resin compositions of each example and a comparative example obtained above (the resin compositions of Examples 7 to 9 and Comparative Example 2) were coated on a PET film with a thickness of 38 μm (manufactured by TEIJIN LIMITED, product name: G2000). Thereafter, they were heated and dried at 140° C. for five minutes to fabricate a resin film in a B-stage state. After the resin film was peeled off from the PET film, the resin powders were obtained by pulverization. A double-side copper foil-attached resin plate was obtained by the same procedure as described above (Examples 1 to 6 and Comparative Example 1) except that the resin powders were used.

### <Method for Measuring Dielectric Properties (Dielectric Constant (Dk) and Dielectric Loss Tangent (Df))>

The double-side copper foil-attached resin plate obtained as described above was immersed in a 10% by mass solution of ammonium persulfate (manufactured by MITSUBISHI GAS CHEMICAL COMPANY, INC.), which is a copper etching solution, to remove the copper foils on both sides, and a test piece having a size of 70 mm×50 mm was fabricated. Next, a test piece immediately after drying at 105° C. for one hour, and a test piece dried at 105° C. for one hour and then left for 24 hours at an ambient temperature of 25° C. and a humidity of 50% RH, each had their dielectric constant (Dk) and dielectric loss tangent (Df) measured at an ambient temperature of 25° C. in 10 GHz bands using a split-post dielectric resonator (SPDR). A vector network analyzer E8364B manufactured by Agilent Technologies, Inc. and a 10 GHz SPDR manufactured by Vega Technology, Inc. were used as measuring devices.

Tables 3 and 4 show results. In Tables 3 and 4, “Dk immediately after drying” and “Df immediately after drying” indicate the dielectric constant (Dk) and dielectric loss tangent (Df) of the test piece immediately after drying at 105° C. for one hour, respectively. Further, “Dk after 24 hr” and “Df after 24 hr” indicate the dielectric constant (Dk) and dielectric loss tangent (Df) of the test piece dried at 105° C. for one hour and then left for 24 hours at an ambient temperature of 25° C. and a humidity of 50% RH, respectively. Still further, “ΔDf (24 hr—immediately after drying)” indicates the difference in dielectric loss tangents (Df) before and after the test piece was left for 24 hours at an ambient temperature of 25° C. and a humidity of 50% RH.

### <Method for Measuring Coefficient of Thermal Expansion>

The double-side copper foil-attached resin plate obtained as described above was immersed in a 10% by mass solution of ammonium persulfate (manufactured by MITSUBISHI GAS CHEMICAL COMPANY, INC.), which is a copper etching solution, to remove the copper foils on both sides, and a test piece having a size of 5 mm square was fabricated. Next, the coefficient of thermal expansion of the test piece at a temperature of 3° C. to 120° C. was measured in accordance with the IPC (The Institute for interconnecting and Packaging Electronic Circuits) standard, using a thermomechanical analyzer (TMA) (manufactured by TA Instruments Japan Inc., product name: Q400).

More specifically, measurements were performed twice under conditions where a temperature was in a range from 30° C. to 260° C., a load was 5 g, and a temperature increase rate was 10° C./min. The coefficient of thermal expansion was the average coefficient of thermal expansion at a temperature of 30° C. to 120° C. in the second measurement. The coefficient of thermal expansion is the coefficient of thermal expansion in the thickness direction of the resin plate. Tables 3 and 4 show results.

### <Method for Measuring Copper Foil Peeling Strength>

The copper foil peeling strength as an index of adhesiveness to a metal foil was measured for Comparative Example 1, Examples 1 to 3, and Example 5.

The double-side copper foil-attached resin plate obtained as described above was processed such that the copper foil was removed by means etching, while leaving a linear portion of 3 mm width, and the copper foil removed resin plate was used as a test piece. The formed linear copper foil was attached on a Compact Tabletop Tester (manufactured by SHIMADZU CORPORATION, product name “EZ-TEST”), and the peeling strength was measured by peeling in a direction of 90 degrees at room temperature (25° C.). The pulling speed for peeling the copper foil was set at 50 mm/min. Table 3 shows results.

TABLE 3

| | | Comparative | | | | | | |
|---|---|---|---|---|---|---|---|---|
| | | Example 1 | Example 1 | Example 2 | Example 3 | Example 4 | Example 5 | Example 6 |
| Compound | | Compound 7 | Compound 1 | Compound 4 | Compound 5 | Compound 2 | Compound 6 | Compound 3 |
| Dielectric | Dk immediately | 2.7 | 2.7 | 2.7 | 2.7 | 2.7 | 2.7 | 2.7 |
| properties | after drying | | | | | | | |
| | Df immediately | 0.0009 | 0.0004 | 0.0005 | 0.0006 | 0.0010 | 0.0005 | 0.0012 |
| | after drying | | | | | | | |
| | Dk after 24 hr | 2.7 | 2.7 | 2.7 | 2.7 | 2.7 | 2.7 | 2.7 |
| | Df after 24 hr | 0.0015 | 0.0007 | 0.0008 | 0.0010 | 0.0015 | 0.0008 | 0.0016 |
| | ΔDf (24 hr − | 0.0006 | 0.0003 | 0.0003 | 0.0004 | 0.0005 | 0.0003 | 0.0004 |
| | immediately | | | | | | | |
| | after drying) | | | | | | | |

| | | | | | | | |
|---|---|---|---|---|---|---|---|
| Coefficient of thermal | 42 | 47 | 37 | 45 | 43 | 41 | 45 |
| expansion (ppm/° C.) | | | | | | | |
| Copper foil peeling | 1.9 | 2.0 | 2.2 | 2.4 | — | 1.7 | — |
| strength (N/cm) | | | | | | | |

TABLE 4
(Unit: parts by mass)

| | | Compar- | | | |
|---|---|---|---|---|---|
| | | ative | | | |
| | | Exam- | Exam- | Exam- | Exam- |
| | | ple 2 | ple 7 | ple 8 | ple 9 |
| Compound 7 | | 75 | | | |
| Compound 1 | | | 75 | | |
| Compound 4 | | | | 75 | |
| Compound 5 | | | | | 75 |
| Maleimide compound | | 25 | 25 | 25 | 25 |
| Dielectric | Dk immediately | 2.7 | 2.7 | 2.7 | 2.6 |
| properties | after drying | | | | |
| | Df immediately | 0.0012 | 0.0010 | 0.0012 | 0.0010 |
| | after drying | | | | |
| | Dk after 24 hr | 2.7 | 2.7 | 2.7 | 2.7 |
| | Df after 24 hr | 0.0025 | 0.0020 | 0.0023 | 0.0018 |
| | ΔDf (24 hr - | 0.0013 | 0.0010 | 0.0011 | 0.0008 |
| | immediately | | | | |
| | after drying) | | | | |

| | | | | |
|---|---|---|---|---|
| Coefficient of thermal | 50 | 50 | 54 | 40 |
| expansion (ppm/° C.) | | | | |

Table 3 revealed that Examples 1 to 6, in which compounds 1 to 6 were used, had a smaller value of ΔDf (24 hr—immediately after drying) than that in Comparative Example 1 in which compound 7 was used. This indicates that deterioration in dielectric properties due to moisture absorption was suppressed.

Further, Table 4 revealed that Examples 7 to 9, in which a resin composition containing compound 1, 4, or 5 was used, had a smaller value of ΔDf (24 hr—immediately after drying) than that in Comparative Example 2 in which compound 7 was used. This indicates that deterioration in dielectric properties due to moisture absorption was suppressed.

1. A compound comprising:
an indene ring;
a vinylbenzyl group; and
an aromatic ring-containing group of 6 or more carbon atoms without a polymerizable carbon-carbon double bond, wherein
both of the vinylbenzyl group and the aromatic ring-containing group are directly bonded to the indene ring.

2. A compound represented by formula (1): 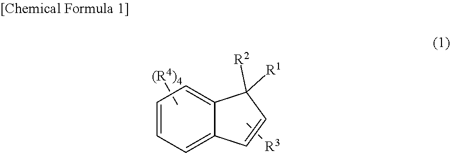
where in formula (1), R¹ to R⁴ each independently represents a hydrogen atom or a monovalent substituent; at least one of R¹, R², or R³ is a vinylbenzyl group, and at least another one of R¹, R², and R³ is an aromatic ring-containing group of 6 or more carbon atoms without a polymerizable carbon-carbon double bond; and all four R⁴s may be different from each other, or two or more of four R⁴s may be identical to each other.

3. The compound according to claim 1, wherein
the aromatic ring-containing group is an unsubstituted aryl group or an aryl group with a substituent without a polymerizable carbon-carbon double bond, or an unsubstituted arylalkyl group or an arylalkyl group with a substituent without a polymerizable carbon-carbon double bond.

4. The compound according to claim 3, wherein
the aromatic ring-containing group is an unsubstituted benzyl group or a benzyl group with a substituent without a polymerizable carbon-carbon double bond.

5. A resin composition comprising:
the compound according to claim 1.

6. A prepreg comprising:
the resin composition according to claim 5, or a semi-cured product of the resin composition.

7. A resin film comprising:
the resin composition according to claim 5, or a semi-cured product of the resin composition.

8. A metal-clad laminate comprising:
a cured product of the resin composition according to claim 5; and
a metal foil.

9. A printed wiring board comprising:
a cured product of the resin composition according to claim 5.

10. A semiconductor package comprising:
the printed wiring board according to claim 9; and
a semiconductor element.

11. A semiconductor package comprising:
a semiconductor element; and
a cured product of the resin composition according to claim 5 for sealing the semiconductor element.

12. A printed wiring board comprising:
at least one of a surface protective film or an interlayer insulating film formed with the resin composition according to claim 5.

13. A resin composition comprising:
the compound according to claim 2.

14. A prepreg comprising:
the resin composition according to claim 13, or a semi-cured product of the resin composition.

15. A resin film comprising:
the resin composition according to claim 13, or a semi-cured product of the resin composition.

16. A metal-clad laminate comprising:
a cured product of the resin composition according to claim 13; and
a metal foil.

17. A printed wiring board comprising:
a cured product of the resin composition according to claim 13.

18. A semiconductor package comprising:
the printed wiring board according to claim 17; and
a semiconductor element.

19. A semiconductor package comprising:
a semiconductor element; and
a cured product of the resin composition according to claim 13 for sealing the semiconductor element.

20. A printed wiring board comprising:
at least one of a surface protective film or an interlayer insulating film formed with the resin composition according to claim 13.
